(19) United States
(12) Patent Application Publication
(10) Pub. No.: US 2010/0065653 A1
(43) Pub. Date: Mar. 18, 2010

(54) WICKS FOR DISPENSERS OF VAPORIZABLE MATERIALS

(76) Inventors: James P. Wingo, Peachtree City, GA (US); Timothy Martin, Newnan, GA (US); Gary Kessinger, Alpharetta, GA (US); Trevor Waghorn, Peachtree City, GA (US)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/534,523

(22) Filed: Aug. 3, 2009

Related U.S. Application Data
(60) Provisional application No. 61/085,542, filed on Aug. 1, 2008.

(51) Int. Cl. A61L 9/12; B23P 11/00; D02G 3/00

(52) U.S. Cl. 239/6; 239/44; 29/428; 428/398

(57) ABSTRACT

The present invention provides wicks for use in vapor dispensing devices which can facilitate manufacture or assembly of the vapor dispensing devices. In one embodiment, a wick of the present invention comprises a porous body, a first end and a second end, the first end having a cross-sectional area less than the cross-sectional area of the body, and the second end having a cross-sectional area less than the cross-sectional area of the body.

### RELATED APPLICATION DATA

The present application claims priority pursuant to 35 U.S.C. §119(e) to U.S. Provisional Patent Application Ser. No. 61/085,542, filed Aug. 1, 2008 which is hereby incorporated by reference in its entirety.

### FIELD OF THE INVENTION

The present invention relates to dispensers of vaporizable materials and, in particular, to wicks for use in dispensers of vaporizable materials.

### BACKGROUND OF THE INVENTION

Vapor dispensing devices, such as air fresheners, are often placed in a room to conceal odors in the room or to add a fragrant scent to the air. The need for effectively combating malodors in homes and enclosed public buildings by odor masking or destruction is well established, as is the dispensing of insect control materials for killing or deterring insects. Various kinds of vapor dispensing devices have been employed for these purposes. In particular, wicking devices are well known for dispensing volatile liquids into the atmosphere, such as a fragrance, deodorant, disinfectant, insect repellant, or insecticide active agent. A typical wicking device utilizes a wick to dispense a volatile liquid from a liquid reservoir. Additionally, wicking devices in which the wicking action is promoted by a heat source are also known.

Many air fresheners are commercially available. Air fresheners that utilize wicking action and are plug-in and/or battery-powered diffusers are particularly popular with consumers. In these devices, a resistance heater is disposed in a housing, out of which electrical prongs extend. When the prongs are plugged into a wall socket, the resistance heater generates heat. A substance, such as a fragrance or an insect repellant, to be emitted into the air is maintained, typically in liquid form, in close proximity to the heater. As the heater heats the substance, controlled amounts of the substance are vaporized and emitted into the surrounding atmosphere. These devices are particularly well suited for domestic use, especially in rooms such as kitchens and bathrooms, because they provide a continuous, controlled flow of a desired substance into the air. Battery-powered diffusers are also well known and function in a substantially similar manner, except that the unit is powered by a consumer-grade battery cell rather than electricity from a wall outlet.

In the assembly of vapor dispensing devices, including air fresheners, a wick can be assembled into a wick holder for subsequent placement in and coupling to a reservoir of the device. The assembly of the wick into the wick holder is generally accomplished using high speed automated equipment. If the wick and the wick holder are not properly aligned during assembly, however, the holder can tear or bend the wick leading to problems in downstream processing and potential failure of the device.

Additionally, in the assembly of vapor dispensing devices, a cap can be screwed onto the reservoir after the wick has been positioned in the reservoir. As with assembling the wick into a holder, the cap can be screwed onto the reservoir using high speed equipment. The cap has a recessed portion into which the wick fits to avoid damage to the wick. If the cap and wick are misaligned, however, screwing the cap onto the reservoir can tear or bend the wick thereby compromising the performance of the vapor dispensing device. Moreover, in some cases, misalignment between the cap and the wick can crush the wick into the reservoir during cap attachment leading to device failure.

Furthermore, amounts of volatized liquid released by plug-in and/or battery powered vapor dispensing devices depends on several considerations including evaporation properties of the liquid, applied heating mechanisms and convection air. In view of these considerations, changing the volatized liquid release profile of a vapor dispensing device is difficult and, in some cases, cost prohibitive. Changing the evaporation properties of a fragrance, for example, can result in a change in fragrance performance and require a complete reformulation of the fragrance. Moreover, altering heating and convection air mechanisms would require redesign of electrical circuitry and housing of a vapor dispensing device.

### SUMMARY

In view of the foregoing disadvantages, the present invention, in one aspect, provides wicks for use in vapor dispensing devices which can facilitate manufacture or assembly of the vapor dispensing devices. In some embodiments, wicks of the present invention can correct for misalignments between the wick and a wick holder during assembly of the wick into the wick holder, thereby reducing the potential for wick tearing and/or bending. Moreover, in some embodiments, wicks of the present invention can also correct for misalignments between the cap and the wick, thereby reducing the potential for wick tearing, bending and/or crushing when the cap is attached to a reservoir containing the wick. Preventing wick degradation and maintaining wick alignment during assembly processes can result in the production of vapor dispensing devices with favorable performance characteristics and can reduce manufacturing inefficiencies resulting from quality control issues associated with defective vapor dispensing devices.

In another aspect, the present invention, in some embodiments, can provide wicks operable to modulate the amount of volatized liquid released from a vapor dispensing device over a period of time. Providing wicks operable to modulate volatized liquid release from a vapor dispensing device, in some embodiments, can obviate the complexities and cost associated with altering liquid evaporation properties, circuit redesign and/or housing redesign.

In one embodiment, a wick of the present invention comprises a porous body, a first end and a second end, the first end having a cross-sectional area less than the cross-sectional area of the body, and the second end having a cross-sectional area less than the cross-sectional area of the body. In some embodiments, the first end or the second end is tapered. In some embodiments, both the first end and the second end of the wick are tapered. In other embodiments, the first end or the second end of the wick is recessed from the perimeter of the body. Moreover, in some embodiments, both the first end and the second end are recessed from the perimeter of the body. In some embodiments, only the first end or the second end of the wick has a cross-sectional area less than the cross-sectional area of the body of the wick.

In some embodiments, the ratio of the cross-sectional area of the first end or the second end of the wick to the cross-sectional area of the wick body ranges from about 0.1 to about 0.9. In other embodiments, the ratio of the cross-sectional area of the first end or the second end of the wick to the cross-sectional area of the wick body ranges from about 0.3 to about 0.7. In some embodiments, the ratio of the cross-sectional area of the first end or the second end of the wick to the cross-sectional area of the wick body ranges from about 0.4 to about 0.6. In some embodiments, where the first end and the second end of the wick are tapered or recessed, the ratios of the cross-sectional areas of the first and second ends to the cross-sectional area of the wick body are selected independently of one another.

The first end or the second end of the wick, according to some embodiments of the present invention, can correct misalignment between the wick and a wick holder during assembly of the wick with the wick holder. In the event the wick and the wick holder are misaligned prior to assembly or engagement, the smaller cross-sectional area of the first or second end of the wick can still fit within the opening of the wick holder. Once in the opening of the wick holder, the first or second end of the wick aligns the body of the wick with the wick holder as the wick is assembled with the wick holder, thereby correcting misalignment between the wick and wick holder. The first or second end of a wick of the present invention aligns the body of the wick with the wick holder whether the wick is pushed into the wick holder or whether the wick holder is pushed over the wick.

As discussed herein, correcting misalignment between the wick and wick holder reduces or precludes wick tearing or bending during assembly of the wick into the wick holder. A wick wherein the ends of the wick have the same or substantially the same cross-sectional area as the body of the wick cannot correct for misalignment between the wick and the wick holder and, as a result, requires accurate alignment with the wick holder in order to avoid tearing or bending.

In some embodiments, misalignment between a wick and a wick holder results from misalignment of a wick pick and place device and the wick holder. In another embodiment, misalignment between a wick and a wick holder results from one or more bends in the body of the wick. In a further embodiment, misalignment between the wick and wick holder results from misalignment of a wick pick and place device and a track transporting wicks from a feeder bowl to the pick and place device. Such a misalignment can cause the wick to be improperly oriented by the pick and place device thereby resulting in misalignment with the wick holder during assembly.

The first end or the second end of a wick of the present invention, in some embodiments, additionally corrects misalignment between the wick and a cap attached to the reservoir of vapor dispensing device. As provided herein, in the assembly of a vapor dispensing device, a cap comprising a recessed portion for receiving a wick is attached to the reservoir of the device after the wick has been positioned in the reservoir. In the event that the wick and the cap are misaligned prior to attachment of the cap, the smaller cross-sectional area of the first or second end of the wick can still fit within the recessed portion of the cap. Once in the recessed portion of the cap, the first or second end of the wick aligns the body of the wick with the recessed portion of the cap as the cap is attached to the reservoir. Correcting misalignment between the wick and the cap reduces or precludes wick tearing, bending, and/or crushing during attachment of the cap to the reservoir of a vapor dispensing device.

Moreover, correcting misalignment between the wick and the cap assists in the proper attachment of the cap to the reservoir. In some embodiments, for example, a cap is screwed onto a reservoir. Misalignment between the cap and reservoir can lead to cross-threading during the screwing process. Correcting such misalignment with the first or second end of a wick of the present invention reduces the potential for cross-threading and other improper cap attachment mechanisms.

In some embodiments, misalignment between a wick and a cap results from misalignment between the reservoir in which the wick is at least partially disposed and the cap. In another embodiment, misalignment between a wick and a cap results from one or more bends in the wick.

In another aspect, the present invention provides a vapor dispensing device comprising a reservoir for holding a liquid and a wick at least partially disposed in the reservoir, the wick comprising a porous body, a first end and a second end, the first end having a cross-sectional area less than the cross-sectional area of the body, and the second end have a cross-sectional area less than the cross-sectional area of the body. In some embodiments, the vapor dispensing device further comprises a wick holder coupled to the opening of the reservoir wherein the wick is assembled to the wick holder. In some embodiments, the vapor dispensing device farther comprises a cap attached to the reservoir, the cap covering the wick.

In some embodiments, the first or second end of a wick of the present invention corrects for misalignment between the wick and the opening of a reservoir of a vapor dispensing device during positioning of the wick in the reservoir. As provided herein, a wick, in some embodiments, is at least partially disposed in a reservoir when a wick holder containing the wick is attached to the opening of the reservoir. In the event the wick and the opening of the reservoir are misaligned prior to attachment of the wick holder to the opening of the reservoir, the smaller cross-sectional area of the first or second end of the wick can still fit within the opening of the reservoir. Once in the opening of the reservoir, the first or second end of the wick aligns the body of the wick with the opening of the reservoir. Correcting misalignment between the wick and the opening of a reservoir reduces or precludes wick tearing, bending, and/or crushing during placement of the wick in the reservoir.

Moreover, as the wick is disposed in a wick holder, alignment of the wick with the opening of the reservoir can additionally align the wick holder with the opening of the reservoir. Alignment of the wick holder with the opening of the reservoir facilitates proper attachment of the wick holder to the opening of the reservoir.

In some embodiments, a vapor dispensing device further comprises a housing coupled to the reservoir, the housing comprising a heating element adjacent to at least a portion of the wick for heating the liquid drawn from the reservoir through the wick. A heating element, according to some embodiments, comprises an electric heating element. In some embodiments, the amount of heat provided by a heating element is adjustable. Housings comprising a heating element suitable for use in some embodiments of the present invention are those described in U.S. patent application Ser. No. 11/805,204.

In some embodiments, the first or the second end of the wick can correct misalignment between the wick and a housing during assembly of a vapor dispensing device. As provided herein, in some embodiments, a reservoir comprising a wick at least partially disposed therein is coupled to a housing in the production of a vapor dispensing device. In some embodiments, the housing comprises an aperture in which the wick is at least partially disposed when the housing is coupled to the reservoir. A heating element, in some embodiments, forms part of or is adjacent to the walls of the aperture for providing heat to the wick.

In the event the wick and the aperture of the housing are misaligned prior to assembly, the smaller-cross sectional area of the first or second end of the wick can still fit within the opening of the aperture. Once in the opening of the aperture, the first or second end of the wick aligns the body of the wick with the aperture as the housing is coupled to the reservoir or other structure of the vapor dispensing device. Correcting misalignment between the wick and the aperture of a housing reduces or precludes wick tearing, bending, and/or crushing during coupling of the housing to the reservoir or other structure of the vapor dispensing device. Moreover, in some embodiments, correcting misalignment between the wick and the aperture of a housing provides proper placement of the wick relative to a heating element in the housing. Proper placement of the wick relative to the heating element facilitates distribution of the proper amount of vaporizable material from the vapor dispensing device.

In some embodiments, misalignment between the wick and the aperture of a housing is caused by misalignment between the reservoir in which the wick is at least partially disposed and the housing. In another embodiment, misalignment between the wick and the aperture of a housing is caused by a bend in the body of the wick.

In another aspect, the present invention provides methods of making a vapor dispensing device. In one embodiment, a method of making a vapor dispensing device comprises providing a wick comprising a porous body, a first end and a second end, the first end having a cross-sectional area less than the cross-sectional area of the body, and the second end having a cross-sectional area less than the cross-sectional area of the body, at least partially disposing the wick in a wick holder, providing a reservoir for holding a liquid, and at least partially disposing the wick in the reservoir. In some embodiments, at least partially disposing the wick in the reservoir comprises coupling the wick holder to an opening of the reservoir. Moreover, in some embodiments, a method of making a vapor dispensing device further comprises coupling a cap to the reservoir wherein the cap covers the wick.

In some embodiments, a method of making a vapor dispensing device further comprises providing a housing comprising heating element and coupling the housing to the reservoir such that the heating element is adjacent to at least a portion of the wick for heating the liquid drawn through the wick.

In some embodiments, methods of making vapor dispensing devices further comprise correcting misalignment of the wick and the wick holder by aligning the wick and the wick holder with the first or second end of the wick. In some embodiments, methods of making vapor dispensing devices further comprise correcting misalignment of the wick and the cap by aligning the wick and the cap with the first or second end of the wick. Correcting misalignments between the wick and the wick holder and/or the wick and the cap by self-aligning mechanisms provided by the design of the wick can increase manufacturing efficiencies of vapor dispensing devices, especially when high speed automated equipment is responsible for the device assembly.

In another aspect, the present invention provides methods of modulating the amount of volatized liquid or material released by a vapor dispensing device. In one embodiment, a method of modulating the amount of volatized liquid or material released from a vapor dispensing device comprises varying the cross-sectional area of at least one end of the wick of the vapor dispensing device. In some embodiments, the cross-sectional area of at least one end of the wick is varied relative to the cross-sectional area of the body of the wick.

In some embodiments, for example, varying the cross-sectional area of at least one end of the wick comprises decreasing the cross-sectional area of the at least one end of the wick relative to the cross-sectional area of the body of the wick. In other embodiments, varying the cross-sectional area of at least one end of the wick comprises increasing the cross-sectional area of the at least one end of the wick relative to the cross-sectional area of the body of the wick.

In some embodiments, a method of modulating the amount of volatized liquid or material released by a vapor dispensing device comprises varying the ratio of the cross-sectional area of at least one end of the wick to the cross-sectional area of the body of the wick. In some embodiments, the ratio of the cross-sectional area of at least one end of the wick to the cross-sectional area of the body of the wick is varied from about 0.1 to about 0.9. In other embodiments, the ratio of the cross-sectional area of at least one end of the wick to the cross-sectional area of the body of the wick is varied from about 0.3 to about 0.7. In some embodiments, the ratio of the cross-sectional area of at least one end of the wick to the cross-sectional area of the body of the wick is varied from about 0.4 to about 0.6.

Moreover, in some embodiments, modulating comprises decreasing the amount of volatized liquid or material released by the vapor dispensing device. In other embodiments, modulating comprises increasing the amount of volatized liquid or material released by the vapor dispensing device.

These and other embodiments are presented in further detail in the detailed description which follows.

### BRIEF DESCRIPTION OF THE DRAWINGS

FIG. 1 is a perspective view of a wick according to one embodiment of the present invention.

FIG. 2 is a perspective view of a wick according to one embodiment of the present invention.

FIG. 3 is a perspective view of a wick according to one embodiment of the present invention.

FIG. 4 is a perspective view of a wick at least partially disposed in a wick holder according to one embodiment of the present invention.

FIG. 5 is a perspective view of wick at least partially disposed in a reservoir of a vapor dispensing device according to one embodiment of the present invention.

FIG. 6 illustrates a cut-away view of a vapor dispensing device according to one embodiment of the present invention.

FIG. 7 illustrates volatized liquid release rates of wicks according to one embodiment of the present invention.

### DETAILED DESCRIPTION

The present invention provides wicks for use in vapor dispensing devices which can facilitate manufacture or assembly of the vapor dispensing devices. In some embodiments, wicks of the present invention can correct for misalignments between the wick and wick holder and/or wick and cap thereby reducing the potential for wick tearing, bending and/or crushing during the assembly of a vapor dispensing device.

Preventing wick degradation and maintaining wick alignment during assembly processes results in the production of vapor dispensing devices with favorable performance characteristics and reduces manufacturing inefficiencies resulting from quality control issues associated with defective vapor dispensing devices. Preventing wick degradation and maintaining wick alignment during assembly processes, in some embodiments, for example, can increase or maintain manufacturing line speeds as the jamming of assembly equipment caused by misaligned wicks is reduced or eliminated. Moreover, in some embodiments, preventing wick degradation reduces the frequency and the degree to which assembly machinery must be cleaned as a result of dust accumulation resulting from pieces of wick sheared during the assembly process.

### Wicks for Use in Vapor Dispensing Devices

In one embodiment a wick of the present invention comprises a porous body, a first end and a second end, the first end having a cross-sectional area less than the cross-sectional area of the body, and the second end having a cross-sectional area less than the cross-sectional area of the body. In some embodiments, the first end or the second end is tapered. In some embodiments, both the first end and the second end of the wick are tapered. In other embodiments, the first end or the second end of the wick is recessed from the perimeter of the body. Moreover, in some embodiments, both the first end and the second end are recessed from perimeter of the body.

In some embodiments, only the first end or the second end of the wick has a cross-sectional area less than the cross-sectional area of the body of the wick.

A wick, according to some embodiments of the present invention, has any desired shape. In some embodiments, a wick has a cylindrical shape. In some embodiments, a wick has the shape of any polygon, including a triangle, parallelogram, pentagon, hexagon, heptagon, octagon, etc.

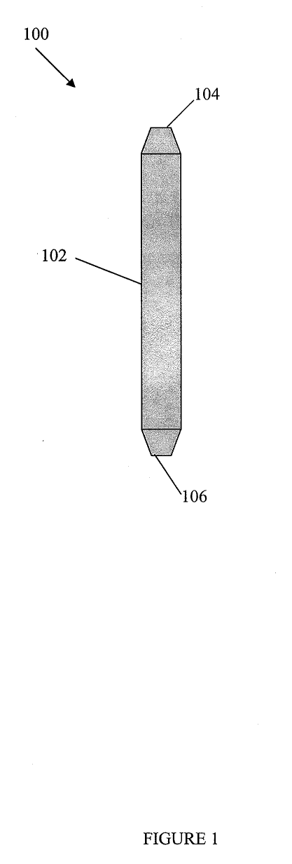

FIG. 1 illustrates a perspective view of a wick according to one embodiment of the present invention. As illustrated in FIG. 1, the wick (100) comprises a body (102), a first end (104) and a second end (106). The first end (104) and the second end (106) of the wick (100) are tapered, each having a cross-sectional area less than the cross-sectional area of the body (102).

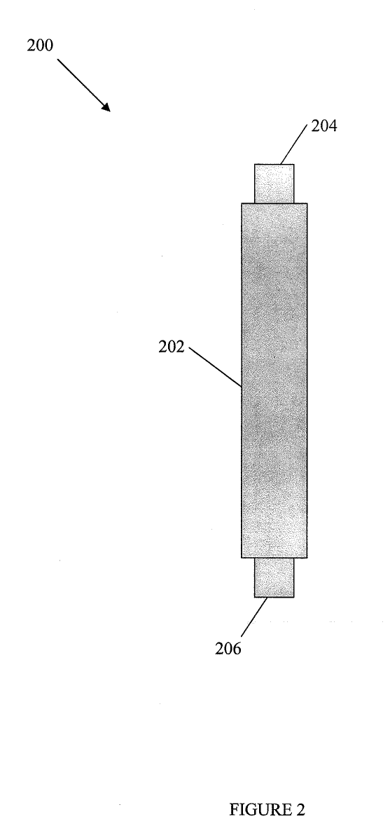

FIG. 2 illustrates a perspective view of a wick according to another embodiment of the present invention. The wick (200) illustrated in FIG. 2 comprises a body (202), a first end (204) and a second end (206). The first end (204) and the second end (206) each have a cross-sectional area less than the cross-sectional area of the body (202). In contrast to being tapered, the first end (204) and the second end (206) are recessed from the perimeter of the body (202) of the wick (200).

In some embodiments, the cross-sectional area of the first end and the cross-sectional area of the second end of a wick is less than the cross-sectional area of the body by any desired amount. Factors governing the cross-sectional areas of the first and second ends of the wick include tolerances imposed by the wick holder and/or cap provided during the assembly of a vapor dispensing device and the desired rate of release of volatized material from the vapor dispensing device.

In some embodiments, the first end and/or the second end of the wick has a cross-sectional area of at least 10% less than the cross-sectional area of the body of the wick. In other embodiments, the first end and/or the second end of the wick has a cross-sectional area of at least 20% less than the cross-sectional area of the body of the wick. In another embodiment, the first end and/or the second end of the wick has a cross-sectional area of at least 30% less than the cross-sectional area of the body of the wick. In a further embodiment, the first end and/or the second end of the wick has a cross-sectional area of at least 50% less than the cross-sectional area of the body of the wick.

In some embodiments, the first end and/or the second end of the wick has a cross-sectional area raging from about 10% to about 90% less than the cross-sectional area of the body of the wick. In some embodiments, the first end and/or the second end of the wick has a cross-sectional area raging from about 20% to about 70% less than the cross-sectional area of the body of the wick. In some embodiments, the first end and/or the second end of the wick has a cross-sectional area raging from about 5% to about 95% less than the cross-sectional area of the body of the wick.

In some embodiments, the first end and the second end of the wick have the same or substantially the same cross-sectional area. In some embodiments wherein the first and second ends of the wick have the same cross-sectional area, orientation of the wick prior to assembly in a wick holder and/or a vapor dispensing device is not required. In other embodiments, the first end and the second end of the wick have different cross-sectional areas.

In some embodiments, the ratio of the cross-sectional area of the first end or the second end of the wick to the cross-sectional area of the wick body ranges from about 0.1 to about 0.9. In other embodiments, the ratio of the cross-sectional area of the first end or the second end of the wick to the cross-sectional area of the wick body ranges from about 0.3 to about 0.7. In some embodiments, the ratio of the cross-sectional area of the first end or the second end of the wick to the cross-sectional area of the wick body ranges from about 0.4 to about 0.6. In some embodiments, where the first end and the second end of the wick are tapered or recessed, the ratios of the cross-sectional areas of the first and second ends to the cross-sectional area of the wick body are selected independently of one another.

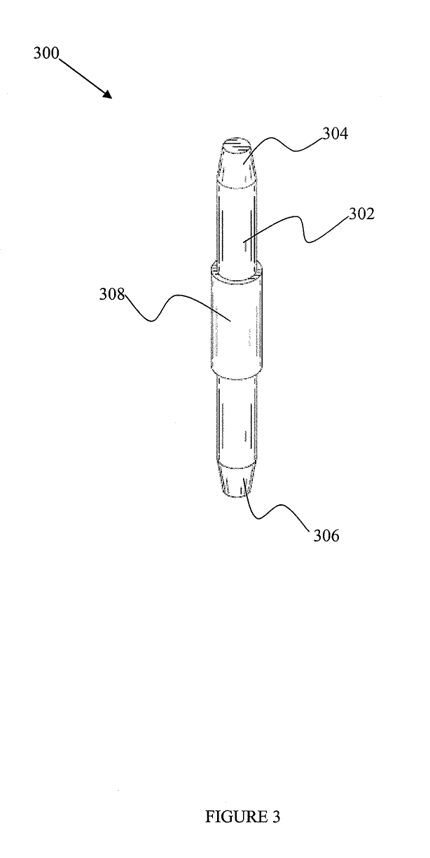

In some embodiments, a wick of the present invention further comprises a collar. FIG. 3 illustrates a wick comprising a collar according to one embodiment of the present invention. As illustrated in FIG. 3, the wick (300) comprises a body (302), a first end (304), a second end (306) and a collar (308). The collar (308) is positioned around the body (302) of the wick (300). The first end (304) and the second end (306) tapered each having a cross-sectional area less than the cross-sectional area of body (302).

A collar is not taken into consideration for the purposes of the present invention in determining whether a first end and/or a second end of a wick has a cross-sectional area less than the cross-sectional area of the body of the wick.

In some embodiments, a collar is co-molded with a wick. In other embodiments, a wick is assembled within the opening of a collar by frictional or other mechanical engagement. In some embodiments, a collar of a wick has a length ranging from about 0.125 to about 1 inch. Additionally, in some embodiments, a collar of a wick has a diameter of up to about 0.5 inch.

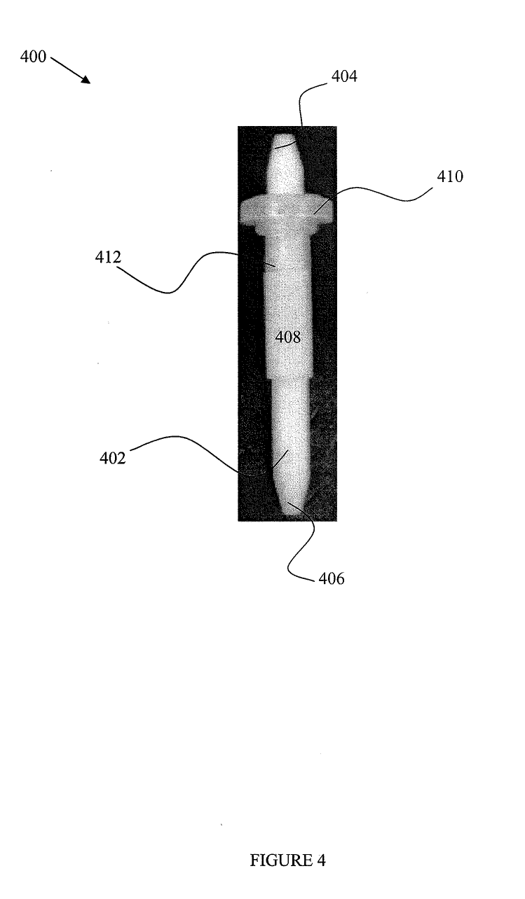

In some embodiments, the collar of a wick facilitates placement of the wick holder along the body of the wick. FIG. 4 illustrates a perspective view of a wick at least partially disposed in a wick holder according to one embodiment of the present invention. The wick (400) illustrated in FIG. 4 comprises a body (402), a first end (404), a second end (406), and collar (408). The first and second ends (404, 406) of the wick (400) each have a cross-sectional area less than the cross-sectional area of the body (402) irrespective of the presence of the collar (408). A wick holder (410) is positioned along the body (402) of the wick (400), wherein the bottom opening (412) of the wick holder (410) abuts the collar (408). The collar (408) acts as a stop for the wick holder (410) during assembly of the wick (400) and the wick holder (410).

Moreover, in the event that the wick (400) and the wick holder (410) are misaligned prior to assembly, the smaller cross-sectional area of the first (404) or second (406) end of the wick (400) can still fit within the opening (412) of the wick holder (410). Once in the opening (412) of the wick holder (410), the first (404) or second (406) end of the wick can assist in self-aligning the body (402) of the wick (400) and the wick holder (410) as the wick (400) is assembled with the wick holder (410), thereby correcting misalignment between the wick (400) and wick holder (410).

The first or second end of a wick of the present invention can assist in self-aligning the body of the wick and the wick holder whether the wick is pushed into the wick holder or whether the wick holder is pushed over the wick.

In some embodiments, a wick of the present invention has an average pore size ranging from about 1 μm to about 500 μm. In another embodiment, a wick of the present invention has an average pore size ranging from about 5 μm to about 200 μm or from about 10 μm to about 150 μm. In some embodiments, a wick has an average pore size ranging from about 30 μm to about 120 μm or from about 15 μm to about 50 μm. In a further embodiment, a wick has an average pore size less than about 1 μm or greater than about 500 μm.

In some embodiments, a wick of the present invention has a porosity ranging from about 10 percent to about 90 percent. In another embodiment, a wick of the present invention has a porosity ranging from about 30 percent to about 60 percent. In some embodiments, a wick has a porosity ranging from about 40 percent to about 50 percent. In a further embodiment, a wick has a porosity less than about 10 percent or greater than about 90 percent.

Wicks of the present invention can comprise any desired material. Factors governing materials suitable for the construction of wicks of the present invention include compatibility with the liquid to be transferred by the wick, wicking rates offered by the material, ease of material processing, material cost, etc. Wicks, in some embodiments of the present invention, comprise porous polymeric materials, including but not limited to, sintered porous polymeric materials.

In some embodiments, sintered polymeric materials of the present invention comprise one or a plurality of plastics. Plastics, as used herein, include flexible plastics and rigid plastics. Flexible plastics, in some embodiments, comprise polymers possessing moduli ranging from about 15,000 N/cm² to about 350,000 N/cm² and/or tensile strengths ranging from about 1500 N/cm² to about 7000 N/cm². Rigid plastics, according to some embodiments, comprise polymers possessing moduli ranging from about 70,000 N/cm² to about 350,000 N/cm² and have tensile strengths ranging from about 3000 N/cm² to about 8500 N/cm².

Plastics suitable for use in sintered polymeric materials of the present invention, in some embodiments, comprise polyolefins, polyamides, polyesters, rigid polyurethanes, polyacrylonitriles, polycarbonates, polyvinylchloride, polymethylmethacrylate, polyvinylidene fluoride, polyethersulfones, polystyrenes, polyether imides, polyetheretherketones, polysulfones, polyethersulfone, polyphenylene oxide, or combinations or copolymers thereof.

In some embodiments, a polyolefin comprises polyethylene, polypropylene, and/or copolymers thereof Polyethylene, in one embodiment, comprises high density polyethylene (HDPE). High density polyethylene, as used herein, refers to polyethylene having a density ranging from about 0.92 g/cm³ to about 0.97 g/cm³. In some embodiments, high density polyethylene has a degree of crystallinity (% from density) ranging from about 50 to about 90. In another embodiment, polyethylene comprises ultrahigh molecular weight polyethylene (UHMWPE). Ultrahigh molecular weight polyethylene, as used herein, refers to polyethylene having a molecular weight greater than 1,000,000.

In some embodiments wherein a wick of the present invention comprises a sintered polymeric material, the wick is produced by providing a plurality of plastic particles in a mold, the mold comprising a cavity having the desired shape of the wick. The plurality of plastic particles are disposed in the mold and sintered to produce a wick of the present invention. Particles of any of the plastics described herein can be sintered into a wick of the present invention.

Plastic particles, in some embodiments, are sintered at a temperature ranging from about 200° F. to about 700° F. In some embodiments, plastic particles are sintered at a temperature ranging from about 300° F. to about 500° F. The sintering temperature, according to embodiments of the present invention, is dependent upon and selected according to the identity of the plastic particles.

Plastic particles, in some embodiments, are sintered for a time period ranging from about 30 seconds to about 30 minutes. In other embodiments, plastic particles are sintered for a time period ranging from about 1 minute to about 15 minutes or from about 5 minutes to about 10 minutes. In some embodiments, the sintering process comprises heating, soaking, and/or cooking cycles. Moreover, in some embodiments, sintering of plastic particles is conducted under ambient pressure (1 atm). In other embodiments, sintering of plastic particles is conducted under pressures greater than ambient pressure.

In another embodiment, a wick comprises a fibrous material. In some embodiments, the fibrous material is sintered. Fibrous materials, according to some embodiments, comprise monocomponent fibers, bicomponent fibers, or combinations thereof. Monocomponent fibers suitable for use in embodiments of the present invention, in some embodiments, comprise polyethylene, polypropylene, polystyrene, nylon-6, nylon-6,6, nylon 12, copolyamides, polyethylene terephthalate (PET), polybutylene terephthalate (TBP), co-PET, or combinations thereof.

Bicomponent fibers suitable for use in wicks, according to some embodiments of the present invention, comprise polypropylene/polyethylene terephthalate (PET); polyethylene/PET; polypropylene/Nylon-6; Nylon-6/PET; copolyester/PET; copolyester/Nylon-6; copolyester/Nylon-6,6; poly-4-methyl-1-pentene/PET; poly-4-methyl-1-pentene/Nylon-6; poly-4-methyl-1-pentene/Nylon-6,6; PET/polyethylene naphthalate (PEN); Nylon-6,6/poly-1,4-cyclohexanedimethyl (PCT); polypropylene/polybutylene terephthalate (PBT); Nylon-6/copolyamide; polylactic acid/polystyrene; polyurethane/acetal; and soluble copolyester/polyethylene. Biocomponent fibers, in some embodiments, comprise those disclosed in U.S. Pat. Nos. 4,795,668; 4,830,094; 5,284,704; 5,509,430; 5,607,766; 5,620,641; 5,633,032; and 5,948,529.

Bicomponent fibers, according to some embodiments of the present invention, have a core/sheath or side by side cross-sectional structure. In other embodiments, bicomponent fibers have an islands-in-the-sea, matrix fibril, citrus fibril, or segmented pie cross-sectional structure. Bicomponent fibers comprising core/sheath cross-sectional structure and suitable for use in embodiments of the present invention are provided in Table I.

TABLE I
Bicomponent Fibers

| Sheath | Core |
|---|---|
| polyethylene (PE) | polypropylene (PP) |
| ethylene-vinyl acetate | polypropylene (PP) |
| copolymer (EVA) | |
| polyethylene (PE) | polyethylene terephthalate (PET) |
| polyethylene (PE) | polybutylene terephthalate (PBT) |
| Polypropylene (PP) | polyethylene terephthalate (PET) |
| Polypropylene (PP) | polybutylene terephthalate (PBT) |
| polyethylene (PE) | Nylon-6 |
| polyethylene (PE) | Nylon-6,6 |
| polypropylene (PP) | Nylon-6 |
| polypropylene (PP) | Nylon-6,6 |
| Nylon-6 | Nylon-6,6 |
| Nylon-12 | Nylon-6 |
| copolyester (CoPET) | polyethylene terephthalate (PET) |
| copolyester (CoPET) | Nylon-6 |
| copolyester (CoPET) | Nylon-6,6 |
| glycol-modified PET (PETG) | polyethylene terephthalate (PET) |
| polypropylene (PP) | poly-1,4-cyclohexanedimethyl (PCT) |
| polyethylene terephthalate (PET) | poly-1,4-cyclohexanedimethyl (PCT) |
| polyethylene terephthalate (PET) | polyethylene naphthalate (PEN) |
| Nylon-6,6 | poly-1,4-cyclohexanedimethyl (PCT) |
| polylactic acid (PLA) | polystyrene (PS) |
| polyurethane (PU) | acetal |

In some embodiments, fibers comprise continuous fibers. In other embodiments, fibers comprise staple fibers. In one embodiment, for example, a fiber of a fibrous material comprises a staple bicomponent fiber. Staple fibers, according to some embodiments, have any desired length. In some embodiments, fibrous materials are woven or non-woven. In one embodiment, a fibrous material is sintered.

In some embodiments, a wick has a length up to about 6 inches. In some embodiments, a wick has a length of at least one inch. In other embodiments, a wick has a length ranging from about 2 inched to about 6 inches. A wick, according to some embodiments, has a length less than about 1 inch or greater than about 6 inches. Moreover, the body of a wick, in some embodiments, has width or diameter of up to about 0.5 inches. In some embodiments, the cross-sectional diameter of a tapered or recessed wick end is at least 0.05 inch.

### Vapor Dispensing Devices

In another aspect, the present invention provides a vapor dispensing device comprising a reservoir for holding a liquid and a wick at least partially disposed in the reservoir, the wick comprising a porous body, a first end and a second end, the first end having a cross-sectional area less than the cross-sectional area of the body, and the second end have a cross-sectional area less than the cross-sectional area of the body. In some embodiments, a portion of the wick not disposed in the reservoir, including the first end or the second end of the wick, is exposed to the surrounding environment for releasing vaporizable liquid or other vaporizable material in the reservoir to the surrounding environment. In some embodiments, a portion of the wick not disposed in the reservoir, including the first end or the second end of the wick, is disposed adjacent to a heating element for heating the liquid drawn through the wick.

Reservoirs of vapor dispensing devices of the present invention contain vaporizable liquid or other vaporizable material and can have any desired shape and volume. A wick of the present invention is at least partially disposed in the reservoir for transporting the vaporizable liquid or other vaporizable material from the reservoir for subsequent distribution to the surrounding environment. In some embodiments, the vapor dispensing device further comprises a wick holder coupled to the opening of the reservoir wherein the wick is assembled to the wick holder as described herein.

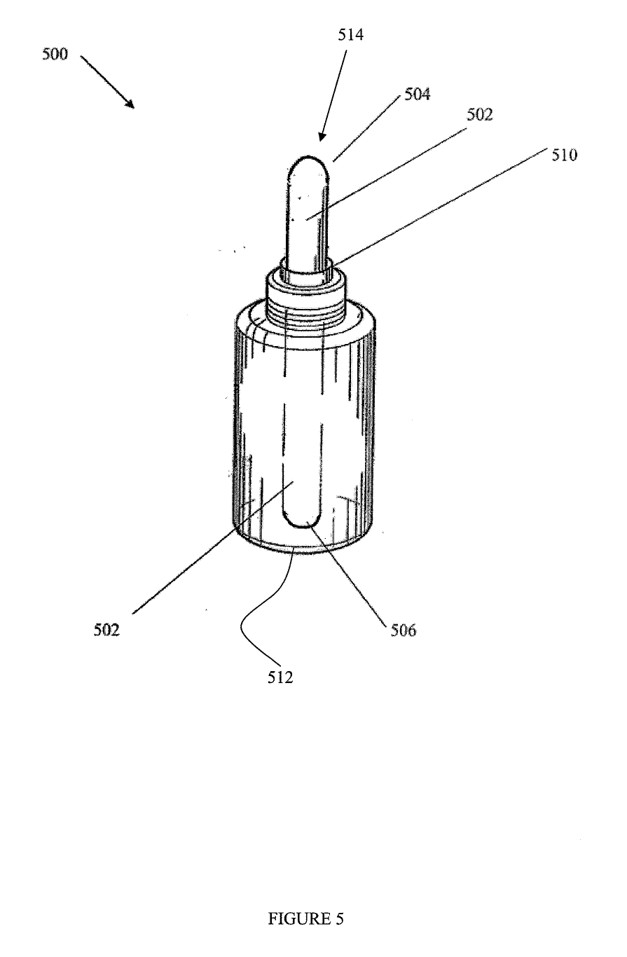

FIG. 5 illustrates a perspective view of wick at least partially disposed in a reservoir of a vapor dispensing device according to one embodiment of the present invention. In the embodiment illustrated in FIG. 5, the vapor dispensing device (500) comprises a reservoir (512) and a wick (514) at least partially disposed in the reservoir (512), the wick (514) comprising a body (502), a first end (504) and a second end (506), the first end (504) and the second end (506) each having a cross-sectional area less than the cross-sectional area of the body (502) of the wick (514). The wick (514) is assembled to a wick holder (510), wherein the wick holder (510) is coupled to the opening of the reservoir (512).

In some embodiments, the vapor dispensing device further comprises a cap attached to the reservoir, the cap covering the portion of the wick not disposed in the reservoir. The cap comprises a recessed portion for receiving the wick when attached to the reservoir. A cap covering the wick of the vapor dispensing device precludes or inhibits premature or undesired release of the vaporizable liquid or material into the surrounding environment.

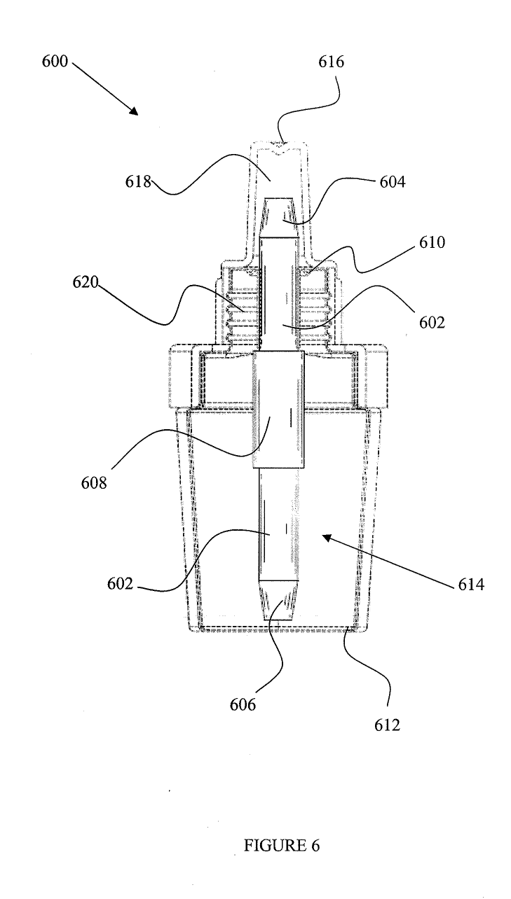

FIG. 6 illustrates a cut away view of a vapor dispensing device having a cap attached to the reservoir of the device. As illustrated in FIG. 6, the vapor dispensing device (600) comprises a wick (614) comprising a body (602), a first end (604) and a second end (606), the first end (604) having a cross-sectional area less than the cross-sectional area of the body (602), and the second end (606) having a cross-sectional area less than the cross-sectional area of the body (602). The wick (614) additionally comprises a collar (608). The wick (614) is assembled to a wick holder (610), wherein the wick holder (610) is coupled to the opening of the reservoir (612). The reservoir (612) is comprises threads (620) proximate the opening, and a cap (616) is attached to the reservoir (612) by engagement with the threads (620). The cap (616) comprises a recessed portion (618) for receiving the wick (614).

Moreover, the first end or the second end of a wick of the present invention, in some embodiments, can additionally correct misalignment between the wick and a cap attached to the reservoir. As provided herein, in the assembly of a vapor dispensing devices, a cap comprising a recessed portion for receiving the wick is attached to the reservoir of the device after the wick has been positioned in the reservoir. In the event that the wick and the cap are misaligned, the smaller cross-sectional area of the first or second end of the wick can still fit within the recessed portion of the cap. Once in the recessed portion of the cap, the first or second end of the wick can assist in self-aligning the body of the wick with the recessed portion of the cap as the cap is attached to the reservoir. Correcting misalignment between the wick and the cap can reduce or preclude wick tearing, bending, and/or crushing during attachment of the cap to the reservoir of a vapor dispensing device.

Moreover, correcting misalignment between the wick and the cap can assist in the proper attachment of the cap to the reservoir. In some embodiments, for example, a cap is screwed onto a reservoir. Misalignment between the cap and reservoir can lead to cross-threading during the screwing process. Correcting such misalignment with the first or second end of a wick of the present invention reduces the potential for cross-threading and other improper attachment mechanisms.

In some embodiments, the first or second end of a wick of the present invention also corrects for misalignment between the wick and the opening of a reservoir of a vapor dispensing device during positioning of the wick in the reservoir. As provided herein, a wick, in some embodiments, is at least partially disposed in a reservoir when a wick holder containing the wick is attached to the opening of the reservoir. In the event the wick and the opening of the reservoir are misaligned prior to attachment of the wick holder to the opening of the reservoir, the smaller cross-sectional area of the first or second end of the wick can still fit within the opening of the reservoir. Once in the opening of the reservoir, the first or second end of the wick aligns the body of the wick with the opening of the reservoir. Correcting misalignment between the wick and the opening of a reservoir reduces or precludes wick tearing, bending, and/or crushing during placement of the wick in the reservoir.

Moreover, as the wick is disposed in a wick holder, alignment of the wick with the opening of the reservoir can additionally align the wick holder with the opening of the reservoir. Alignment of the wick holder with the opening of the reservoir facilitates proper attachment of the wick holder to the opening of the reservoir.

In some embodiments, a vapor dispensing device further comprises a housing coupled to the reservoir. In some embodiments, the housing comprises a heating element adjacent to at least a portion of the wick for heating the liquid drawn through the wick. A heating element, according to some embodiments, comprises a electric heating element.

In some embodiments, the first or the second end of the wick can correct misalignment between the wick and a housing during assembly of a vapor dispensing device. As provided herein, in some embodiments, a reservoir comprising a wick at least partially disposed therein is coupled to a housing in the production of a vapor dispensing device. In some embodiments, the housing comprises an aperture in which the wick is at least partially disposed when the housing is coupled to the reservoir. A heating element, in some embodiments, forms part of or is adjacent to the walls of the aperture for providing heat to the wick.

In the event the wick and the aperture of the housing are misaligned prior to assembly, the smaller-cross sectional area of the first or second end of the wick can still fit within the opening of the aperture. Once in the opening of the aperture, the first or second end of the wick aligns the body of the wick with the aperture as the housing is coupled to the reservoir or other structure of the vapor dispensing device. Correcting misalignment between the wick and the aperture of a housing reduces or precludes wick tearing, bending, and/or crushing during coupling of the housing to the reservoir or other structure of the vapor dispensing device. Moreover, in some embodiments, correcting misalignment between the wick and the aperture of a housing provides proper placement of the wick relative to a heating element in the housing. Proper placement of the wick relative to the heating element facilitates distribution of the proper amount of vaporizable material from the vapor dispensing device.

In some embodiments, a vaporizable material of a vapor dispensing device of the present invention is a liquid. In other embodiments, a vaporizable material is a gel, paste, or a solid such as, but not limited to, a wax. Vaporizable materials, in some embodiments of the present invention, comprise fragrances. In another embodiment, vaporizable materials comprise deodorants, disinfectants, insect repellants, or insecticide active agents.

In some embodiments wherein a vaporizable material is a gel, the gel is constructed by mixing a fragrance, deodorant, disinfectant, insect repellant, and/or insecticide agent with an aqueous based solution and a gel forming agent, such as carrageenan and/or carboxymethylcellulose (CMC). In another embodiment, a fragrance, deodorant, disinfectant, and/or insecticide is mixed with an alcohol based solution and a gel foaming agent in the production of a vaporizable gel material.

Additionally, in some embodiments wherein a vaporizable material is a solid, the solid is constructed by mixing a fragrance, deodorant, disinfectant, insect repellant, and/or insecticide with a liquid wax and subsequently cooling the mixture to solid form. In one embodiment, the mixture is sprayed prior to cooling to faun a powder. Waxes suitable for use in solid vaporizable materials can comprise a natural wax, such as hydroxystearate wax, or a petroleum based wax, such as a paraffin. In some embodiments, polyethylene oxide (PEO) is used as a substrate for a fragrance, deodorant, disinfectant, insect repellants and/or insecticide.

Vaporizable fragrances, disinfectants, deodorants, insect repellants, and insecticides are well known to one of skill in the art and are available from a variety of commercial sources. Common fragrances comprise citrus oils, fruity floral oils, herbal floral oils, lemon oils, orange oils, or combinations thereof. Disinfectants, in some embodiments, comprise denatonium benzoate, hinokitiol, benzthiazolyl-2-thioalkanoic nitriles, alkyl dimethylbenzyl ammonium chlorides, or trichlosan. Insect repellants, in some embodiments, comprise N,N-diethyl-meta-toluamide, citronella oils, or camphor. Additionally, insecticides, in some embodiments, comprise imiprotrin, cypermethrin, bifentrint, or pyrethrins.

Vaporizable materials, in some embodiments, are disposed in a reservoir of the dispenser. In one embodiment, a vaporizable material comprises a liquid. As described herein, a liquid vaporizable material can be transported from the reservoir through the wick for subsequent vaporization or evaporation. In some embodiments vaporization and evaporation is facilitated or accelerated by a heating element adjacent to the wick. In other embodiments, a vaporizable material is disposed on a surface of the wick or otherwise impregnated into the wick. In such embodiments, the wick can serve as the reservoir for the vaporizable material. In one embodiment, for example, a wick is impregnated and/or coated with a solid vaporizable material, such as a wax. In another embodiment, a wick is impregnated and/or coated with a vaporizable material comprising a gel or paste. In some embodiments wherein the wick is impregnated and/or coated with a solid, gel, or paste vaporizable material, the wick serves as a reservoir for the solid, gel, or paste vaporizable material.

### Methods of Making Vapor Dispensing Devices

In another aspect, the present invention provides methods of making a vapor dispensing device. In one embodiment, a method of making a vapor dispensing device comprises providing a wick comprising a porous body, a first end and a second end, the first end having a cross-sectional area less than the cross-sectional area of the body, and the second end having a cross-sectional area less than the cross-sectional area of the body, at least partially disposing the wick in a wick holder, providing a reservoir for holding a liquid, and at least partially disposing the wick in the reservoir. In some embodiments, at least partially disposing the wick in the reservoir comprises coupling the wick holder to an opening of the reservoir. Moreover, in some embodiments, a method of making a vapor dispensing device comprises coupling a cap to the reservoir wherein the cap covers the wick.

In some embodiments, a method of making a vapor dispensing device further comprises providing a housing comprising heating element and coupling the housing to the reservoir such that the heating element is adjacent to at least a portion of the wick for heating the liquid drawn through the wick.

In some embodiments, methods of making a vapor dispensing device further comprise correcting misalignment of the wick and the wick holder by aligning the wick and the wick holder with the first or second end of the wick. In some embodiments, methods of making a vapor dispensing device further comprise correcting misalignment of the wick and the cap by aligning the wick and the cap with the first or second end of the wick. Correcting misalignments between the wick and the wick holder and/or the wick and the cap by self-aligning mechanisms provided by the design of the wick can increase manufacturing efficiencies of vapor dispensing devices, especially when high speed automated equipment is responsible for the device assembly.

### Methods of Modulating Vaporizable Liquid or Material Released

In another aspect, the present invention provides methods of modulating the amount of volatized liquid or material released by a vapor dispensing device. In one embodiment, a method of modulating the amount of volatized liquid or material released from a vapor dispensing device comprises varying the cross-sectional area of at least one end of the wick of the vapor dispensing device. In some embodiments, the cross-sectional area of at least one end of the wick is varied relative to the cross-sectional area of the body of the wick.

In some embodiments, for example, varying the cross-sectional area of at least one end of the wick comprises decreasing the cross-sectional area of the at least one end of the wick relative to the cross-sectional area of the body of the wick. In some embodiments, a linear or substantially linear relationship exists between the amount by which the cross-sectional area of at least one end of the wick is decreased and the change in the amount of volatized liquid released from the vapor dispensing device.

In other embodiments, varying the cross-sectional area of at least one end of the wick comprises increasing the cross-sectional area of the at least one end of the wick relative to the cross-sectional area of the body of the wick.

In some embodiments, a method of modulating the amount of volatized liquid or material released by a vapor dispensing device comprises varying the ratio of the cross-sectional area of at least one end of the wick to the cross-sectional area of the body of the wick. In some embodiments, the ratio of the cross-sectional area of at least one end of the wick to the cross-sectional area of the body of the wick is varied from about 0.1 to about 0.9. In other embodiments, the ratio of the cross-sectional area of at least one end of the wick to the cross-sectional area of the body of the wick is varied from about 0.3 to about 0.7. In some embodiments, the ratio of the cross-sectional area of at least one end of the wick to the cross-sectional area of the body of the wick is varied from about 0.4 to about 0.6.

Moreover, in some embodiments, modulating comprises decreasing the amount of volatized liquid or material released by the vapor dispensing device. In other embodiments, modulating comprises increasing the amount of volatized liquid or material released by the vapor dispensing device.

Aspects of the present invention are further illustrated in the following non-limiting examples.

### Example 1

### Production of a Wick for a Vapor Dispensing Device

HDPE particles having an average size of 238 μm were disposed in a mold and sintered at a temperature of 170° C. for about 3 minutes to produce a wick comprising a porous body, a first end and a second end, the first end having a cross-sectional area about 50% less than the cross-sectional area of the body, and the second end having a cross-sectional area about 50% less than the cross-sectional area of the body. The wick had an average pore size of about 78 μm and a porosity of about 50%. Moreover, the wick had an overall length of about 3 inches. In order to facilitate placement of a wick holder as provided herein, the wick additionally comprised a collar. The collar was co-molded with the body of the wick during the sintering process.

### Example 2

### Production of a Wick for a Vapor Dispensing Device

UHMWPE particles having an average size of 190 μm were disposed in a mold and were sintered at a temperature of 180° C. for about 5 minutes to produce a wick comprising a porous body, a first end and a second end, the first end having a cross-sectional area about 50% less than the cross-sectional area of the body, and the second end having a cross-sectional area about 50% less than the cross-sectional area of the body. The wick had an average pore size of about 26 μm and a porosity of about 60%. Moreover, the wick had an overall length of about 3 inches. In order to facilitate placement of a wick holder as provided herein, the wick additionally comprised a collar. The collar was co-molded with the body of the wick during the sintering process.

### Example 3

### Production of a Vapor Dispensing Device

A vapor dispensing device was produced according to the following procedure. The wick of Example 1 was assembled with a wick holder. The wick holder was pushed over the first or second end of the wick and seated against the collar of the wick. The wick was subsequently at least partially disposed in a reservoir by coupling wick holder to the opening of the reservoir. The reservoir contained a vaporizable liquid in contact with the wick.

The reservoir having the wick at least partially disposed therein was then coupled to a housing, the housing comprising an electric heating element. A portion of the wick not within the reservoir was positioned adjacent to the heating element of the housing.

### Example 4

### Modulating the Amount of Volatized Liquid Released by a Vapor Dispensing Device

A series of wicks comprising HDPE and having the properties listed in Table 2 were produced in accordance with the procedure set forth in Example 1.

TABLE 2
Properties of Wicks

| | Wick First End - | Wick Second End - | | |
|---|---|---|---|---|
| | Percent Cross- | Percent Cross- | | |
| | Sectional | Sectional | Average | |
| Wick | Area of Body | Area of Body | Pore Size | Porosity |
| 1 | 50 | 50 | 80 | 45 |
| 2 | 70 | 70 | 80 | 45 |
| 3 | 90 | 90 | 80 | 45 |
| 4 | 100 | 100 | 80 | 45 |

Wicks 1 through 3 in Table 2 comprise first and second ends each having a cross-sectional diameter less than the cross-sectional diameter of the body of the wick in accordance with embodiments of the present invention. The first and second ends of Wick 1, for example, each have a cross-sectional area of 50% of the cross-sectional area of the body of the wick. The first and second ends of Wick 2 each have a cross-sectional area of 70% of the cross-sectional area of the wick while the first and second ends of Wick 3 each have a cross-sectional area of 90% of the cross-sectional area of the body of the wick. Wick 4 is a comparative example wherein the first and second ends of the wick have the same or substantially the same cross-sectional area as the body of the wick.

Wicks 1 through 4 were at least partially disposed in a reservoir and subsequently coupled to a housing in the production of a vapor dispensing device according to the procedure provided in Example 3. The vapor dispensing devices were weighed and plugged into 110 V AC outlets. The devices were weight at periodic intervals to determine the amount of liquid volatized and released from each device.

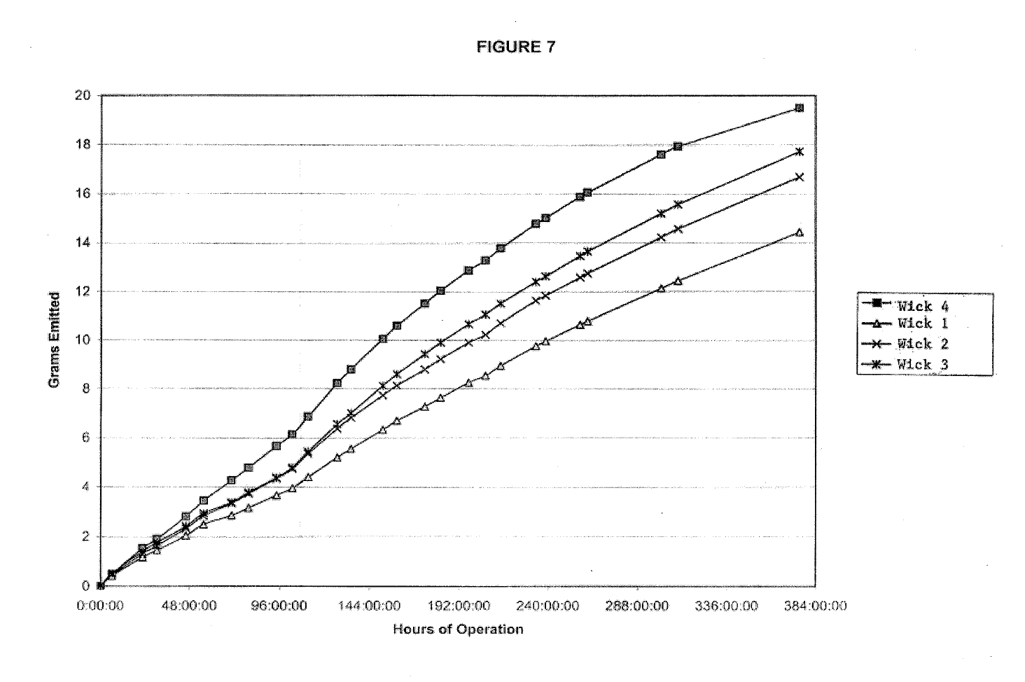

FIG. 7 illustrates the volatized liquid release rates of Wicks 1-4. As provided in FIG. 7, Wick 4 released the most amount of volatized fragrance followed by Wick 3 and Wick 2. Wick 1 having the greatest reduction in cross-sectional area of the wick ends provided the least amount of volatized fragrance release. As a result, the embodiment of the present example demonstrates modulating the amount of volatized liquid, fragrance or material released by a vapor dispensing device by varying the cross-sectional area of at least one end of a wick of the vapor dispensing device.

All patents, patent applications, publications, and abstracts cited above are incorporated herein by reference in their entirety. Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention.

That which is claimed is:

1. A wick of a vapor dispensing device comprising:
a porous body, a first end and a second end, the first end having a cross-sectional area less than the cross-sectional area of the porous body, and the second end having a cross-sectional area less than the cross-sectional area of the porous body.

2. The wick of claim 1, wherein the first end has a cross-sectional area at least 10% less than the cross-sectional area of the porous body.

3. The wick of claim 1, wherein the first end has a cross-sectional area at least 50% less than the cross-sectional area of the porous body.

4. The wick of claim 1 further comprising a collar coupled to the porous body.

5. The wick of claim 1, wherein the porous body comprises sintered plastic particles.

6. The wick of claim 5, wherein the plastic particles comprise a polyethylene, polypropylene, polyamide, polyester, polyurethane, polyacrylonitrile, polycarbonate, polyvinylchloride, polymethylmethacrylate, polyvinylidene fluoride, polyether imide, polyetheretherketone, polysulfone, polyethersulfone, polyphenylene oxide or copolymers or mixtures thereof.

7. A vapor dispensing device comprising:
a reservoir for holding a liquid; and
a wick comprising a porous body, a first end and a second end, the first end having a cross-sectional area less than the cross-sectional area of the porous body, and the second end having a cross-sectional are less than the cross-sectional area of the porous body, wherein the wick is at least partially disposed in the reservoir through an opening in the reservoir.

8. The vapor dispensing device of claim 7, wherein the first end of the wick has a cross-sectional area at least 10% less than the cross-sectional area of the porous body.

9. The vapor dispensing device of claim 7, wherein the first end of the wick has a cross-sectional area at least 50% less than the cross-sectional area of the porous body.

10. The vapor dispensing device of claim 7, wherein the wick is disposed in a wick holder.

11. The vapor dispensing device of claim 7 further comprising a collar coupled to the porous body.

12. The vapor dispensing device of claim 7, wherein the liquid comprises a fragrance, deodorant, disinfectant, insect repellant or insecticide or combinations thereof.

13. The vapor dispensing device of claim 7 further comprising a heating element adjacent to at least a portion of the wick.

14. A method of making a vapor dispensing device comprising:
providing a wick comprising a porous body, a first end and a second end, the first end having a cross-sectional area less than the cross-sectional area of the body, and the second end having a cross-sectional area less than the cross-sectional area of the body;
at least partially disposing the wick in a wick holder;
providing a reservoir for holding a liquid; and
at least partially disposing the wick in the reservoir.

15. The method of claim 14, further comprising correcting misalignment of the wick and the wick holder by aligning the wick and wick holder with the first end or second end of the wick.

16. The method of claim 14, further comprising correcting misalignment of the wick and the reservoir by aligning the wick and the reservoir with the first end or second end of the wick.

17. The method of claim 14, further comprising coupling a cap to the reservoir wherein the cap covers the wick.

18. The method of claim 17, further comprising correcting misalignment of the wick and the cap by aligning the wick and the cap with the first end or second end of the wick.

19. The method of claim 14, wherein the first end of the wick has a cross-sectional area at least 10% less than the cross-sectional area of the body of the wick.

20. A method of modulating an amount of volatized liquid released by a vapor dispensing device having a wick comprising:
varying the cross-sectional area of at least one end of a wick of the vapor dispensing
device.

21. The method of claim 20, further comprising varying the cross-sectional area of both ends of the wick of the vapor dispensing device.

22. The method of claim 20, wherein varying the cross-sectional area of the at least one end of the wick comprises decreasing the cross-sectional area of the at least one end.

23. The method of claim 20, wherein varying the cross-sectional area of the at least one end of the wick comprises increasing the cross-sectional area of the at least one end.

24. The method of claim 20, wherein the amount of volatized liquid released is increased.

25. The method of claim 20, wherein the amount of volatized liquid released is decreased.

26. The method of claim 20, further comprising heating a portion of the wick.
